(19) United States
(12) Patent Application Publication
(10) Pub. No.: US 2004/0176722 A1
(43) Pub. Date: Sep. 9, 2004

(54) Single-use syringe

(76) Inventors: David Francis Capes, Singapore (SG); Patrick Chng Hang Khiang, Singapore (SG); Steven Lau Choon Meng, Singapore (SG); Hiang Seng Low, Singapore (SG)

Correspondence Address: DAVID W. HIGHET, VP AND CHIEF IP COUNSEL, BECTON, DICKINSON AND COMPANY, 1 BECTON DRIVE, MC 110, FRANKLIN LAKES, NJ, 07417-1880, US

(21) Appl. No.: 10/379,960

(22) Filed: Mar. 5, 2003

(51) Int. Cl. A61M 5/00

(52) U.S. Cl. 604/110; 604/218

(57) ABSTRACT

A single-use syringe comprises a barrel having a proximal end, a distal end and an inside surface, and a plunger rod having a proximal portion and a distal portion connected by a disengageable connection. The plunger rod further includes a stopper on its distal end. The disengageable connection is strong enough to hold the proximal and distal portions together during normal use of the syringe and disengageable upon application of an additional force applied to the proximal portion. A catch on the proximal portion of the plunger rod and a discontinuity in the chamber engage to prevent removal of the proximal portion from the barrel.

### FIELD OF THE INVENTION

The present invention relates to a syringe having a disengageable connection on the plunger rod and plunger rod retaining structure to prevent further use of the syringe after fluid delivery or injection.

### BACKGROUND

Throughout the world the re-use of hypodermic syringe products which are intended for single-use only is instrumental in drug abuse and in the transfer of contagious diseases. Intravenous drug users who routinely share and re-use syringes are a high-risk group with resep0ct to the AIDS virus. Also, the effects of multiple use are a major concern in some countries where the repeated use of syringe products during mass inoculation programs may be responsible for the spread of many diseases. Syringes are often recycled in developing countries without proper sterilization.

Many attempts have been made to remedy this problem. Some designs involve the inclusion of structure which will allow the destruction or defeating of the syringe function through a conscious act by the user, such as breaking a syringe or one of its components. In addition, there are single-use hypodermic syringes which become incapable of further use automatically upon delivery of the medication without any additional act on the part of the user.

### SUMMARY OF THE INVENTION

A single-use syringe comprises a barrel and a plunger rod. The barrel includes a fluid chamber having an inside surface, an open proximal end, and a distal end having a passageway therethrough in fluid communication with the chamber. The plunger rod includes a longitudinal axis, a proximal portion and a distal portion connected by a disengageable connection. The distal portion also includes a stopper slidably positioned in fluid-tight engagement with the inside surface of the chamber for drawing fluid into and out of the chamber by movement of the plunger rod relative to the barrel. The disengageable connection is strong enough to hold the proximal portion and the distal portion together during normal use of the syringe and disengageable upon application of an additional force applied to the proximal portion along the longitudinal axis. A catch on the proximal portion of the plunger rod and a discontinuity in the chamber are positioned so that upon further distal movement of the proximal portion of the plunger rod, after disengagement of the disengageable connection, the catch engages the discontinuity to lock the proximal portion of the plunger rod in the barrel.

The disengageable connection may include one of said proximal portion and said distal portion having an axial projection including at least one transverse protuberance projecting therefrom. Said protuberance being connected to the other of the proximal portion and the distal portion. The protuberance is or includes a breakable connection. The axial projection may include a plurality of transverse protuberances.

The disengageable connection may comprise one of the proximal and distal portions including a recess and the other of the proximal and distal portions including a projection releasably engaged in the recess in a snap-fit arrangement.

The syringe of the present invention may also include a second disengageable connection on the proximal portion of the plunger rod, proximal to the catch. The second disengageable connection is strong enough to hold the proximal portion of the plunger rod together during normal use of the syringe and disengageable upon application of an additional force along the longitudinal axis to dislodge the plunger rod from the barrel.

The syringe may further include an elongate distal tip extending from the distal end of the barrel wherein the passageway extends through the tip. The syringe may also include a cannula having a proximal end, a distal end and a lumen therethrough with the proximal end of the cannula being joined to the distal end of the barrel so that the lumen is in fluid communication with the passageway.

### BRIEF DESCRIPTION OF THE DRAWINGS

FIG. 1 is an exploded perspective view of the plunger rod and barrel of a syringe of the present invention.

FIG. 2 is a perspective view of a syringe of the present invention,

FIG. 3 is a partial cross-sectional view of the syringe of FIG. 2 taken along line 3-3.

FIG. 4 is an enlarged cross-sectional view showing the distal end of the syringe barrel and the plunger rod of FIG. 3.

FIG. 5 is a cross-sectional view of the syringe of FIG. 3 illustrating the position of the plunger rod after the disengagement of the disengageable connection.

FIG. 6 is an enlarged cross-sectional view of the distal end of the syringe and plunger rod of FIG. 5.

FIG. 7 is a cross-sectional view of the syringe of FIG. 5 showing the disengagement of the second disengageable connection on the plunger rod.

FIG. 8 is an enlarged side elevational view of a plunger rod of the present invention.

FIG. 9 is a partially cross-section side elevational view of an alternative syringe of the present invention.

FIG. 10 is an enlarged cross-sectional view of the distal end of the barrel and plunger rod of the syringe of FIG. 9.

### DETAILED DESCRIPTION

While this invention is satisfied by embodiments in many different forms, there is shown in the drawings and will herein be described in detail preferred embodiments of the invention with the understanding that the present disclosure is to be considered as exemplary of the principles of the invention and not intended to limit the invention to the embodiments illustrated. The scope of the invention will be measured by the appended claims and their equivalents.

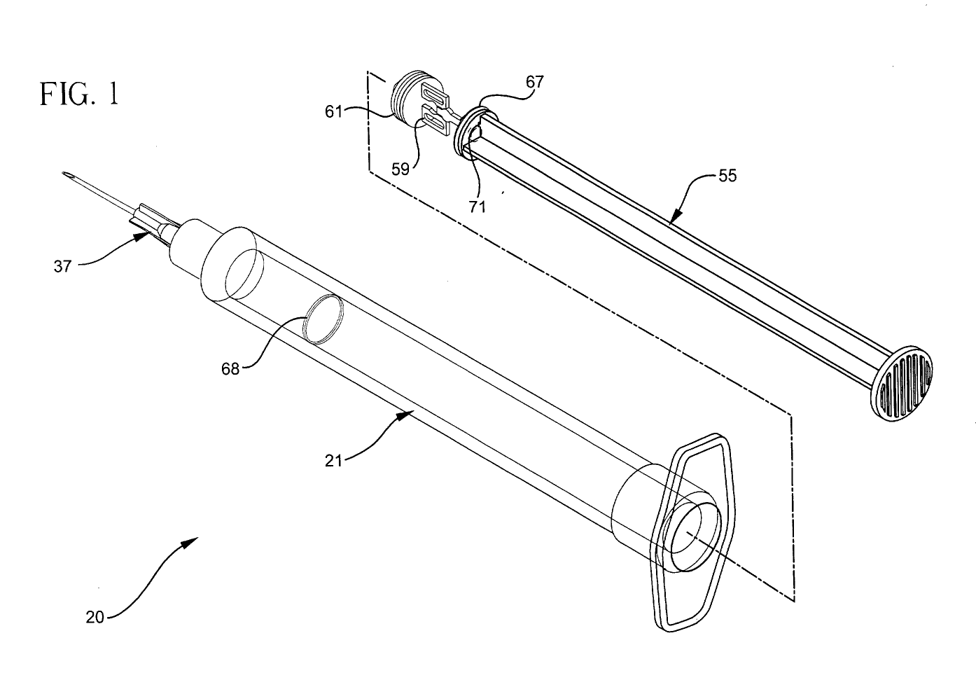

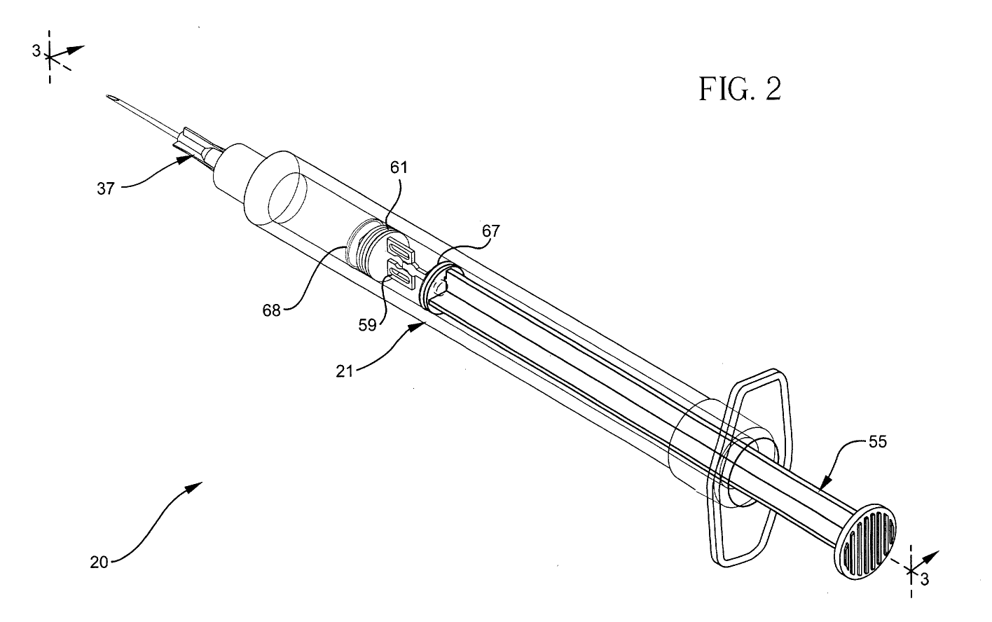

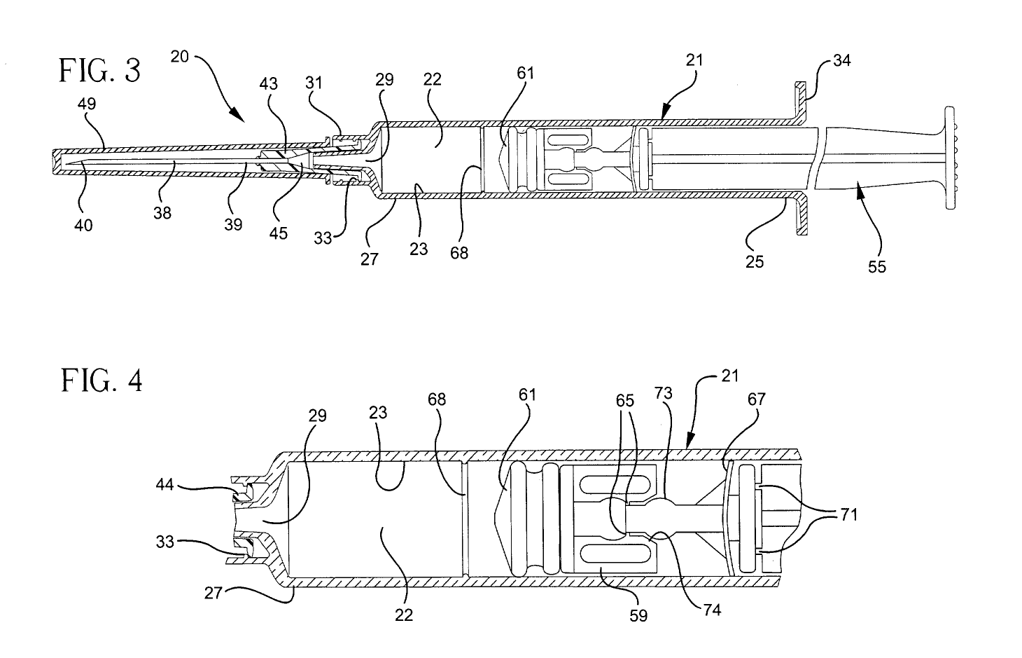

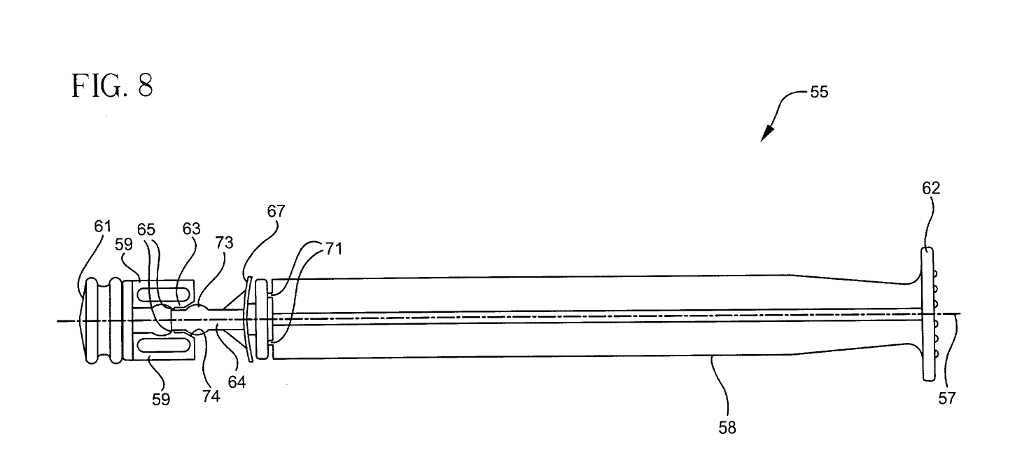

Referring to FIGS. 1-8, a syringe 20 of the present invention includes a barrel 21 having a fluid chamber 22 with an inside surface 23, a proximal end 25, a distal end 27 and an elongate tip 28 extending from the distal end and having a passageway 29 therethrough in fluid communication with the chamber. In this embodiment the elongate tip is preferably frusto-conically shaped. Barrel 21 may also include a collar 31 having an inside surface 32 and at least one thread 33 to engage a needle hub.

The syringe of the present invention is intended to be used with a needle assembly or a permanently attached needle. In this embodiment, needle assembly 37 includes a cannula 38 having a proximal end 39, a distal end 40 and a lumen 41 therethrough. The needle assembly also includes a hub 43 having a proximal open end 44 with a cavity 45 therein, and a distal end 46 joined to proximal end 39 of cannula 38 so that lumen 41 is in fluid communication with cavity 45. A removable needle shield 49 is provided to protect the cannula before use. It is within the purview of the present invention to include needle assemblies having one-piece construction wherein the cannula and the hub are formed of one piece.

The needle assembly with the hub having outwardly extending projections 47 is placed on the distal end of the barrel by aligning the distal tip of the barrel with the cavity in the hub and moving the needle assembly towards the barrel so that the outward projections of the hub engage the thread in the collar of the barrel. The needle assembly is then rotated or screwed into the collar so that the needle assembly is held tightly on the distal tip of the barrel through the interaction of the collar thread and the projections on the hub. This is an excellent structure for many applications since it allows for attaching an appropriately sized needle assembly at the time of use and also allows for using another size needle assembly during filling procedures.

The syringe of the present embodiment includes a plunger rod 55 having a longitudinal axis 57, a proximal portion 58, a distal portion 59 and a stopper 61 on the distal portion. The stopper is slidably positioned in fluid-tight engagement with the inside surface of the chamber for drawing fluid into and out of the chamber by movement of the plunger rod relative to the barrel. The plunger rod extends outwardly from proximal end 25 of the barrel. The plunger rod is accessible outside of the proximal end of the barrel and is provided to move the stopper along the barrel to force fluid into and out of the chamber 22 through passageway 29. Plunger rod flange 62 is provided as a convenient structure for applying force to move the plunger rod with respect to the barrel. A flange 34 is provided at the proximal end of the barrel to facilitate handling and for maintaining a relative position of the barrel with respect to the plunger rod during fluid transfer using known procedures.

It is within the purview of the present invention to include plunger rods and stoppers which are separately formed or integrally formed of the same material or different materials such as in two-shot molding, or separately formed of the same or different materials and joined together by mechanical means, adhesives, ultrasonic welding, heat sealing or other suitable means. Stoppers can be made of clastomeric material such as natural rubber, synthetic rubber, thermoplastic elastomers and combinations thereof. Stoppers may also be made of a more rigid material wherein the fluid-tight engagement between the inside surface of the chamber and the stopper is facilitated by flexure of the inside surface. It is understood that the plunger of the present embodiment is merely illustrative of these many possibilities.

Proximal portion 58 and distal portion 59 of the plunger rod are connected by a disengageable connection 63 which is strong enough to hold proximal portion 58 and distal portion 59 together during normal use of the syringe and is disengageable upon application of additional force to the proximal portion.

In this embodiment, the disengageable connection comprises one or more breakable connections connecting the proximal and distal portions of the plunger rod. Specifically, proximal portion 58 includes a distal projection 64 having at least one transverse protuberance projecting therefrom. In this embodiment, there are two transverse protuberances 65. The protuberances are connected to distal portion 59 and are breakable. The distal projection may be circularly shaped and fit into a cylindrically shaped recess in the distal portion. With this cylindrical structure a single protuberance extending up to 360 degrees may be used.

In this embodiment, proximal portion 58, distal portion 59 and protuberances 65 are integrally molded of plastic material. A wide variety of plastic materials are suitable for molding the plunger rod with polystyrene, polypropylene and polyethylene being preferred. It is important to control the modulus of elasticity of material selected for the protuberances which are part of the disengageable connection between the proximal portion and the distal portion of the plunger rod. The disengageable connection must break or fail upon the application of an additional force at the completion of the injection process. If the modulus is too high the break will occur too easily causing premature breakage. If the modulus is too low the breakable connection may not break without the use of excessive force. Also, within the range of acceptable materials and configurations, it is preferred that there be an audible click upon breaking to confirm to the user that breaking has occurred.

The protuberance does not have to be uniform along its length but may include areas of reduced cross section anywhere along its length to enhance its ability to break under the desired conditions. The protuberance may be very short and may be made entirely of adhesive or frangible material. The connection can also be made using a shear pin passing through distal projection 64 and distal portion 59. The shear pin may be made of plastic with one or more notches or stress risers suitably placed to allow breaking at the desired force levels. A disengageable connection between the proximal portion and distal portion may also be accomplished by using a snap-fit arrangement relying on deflection of the distal end or proximal portions to accomplish the separation thereof. In this latter situation, the distal portion and the proximal portion can be individually molded and snapped together during the assembly process. Also, the distal portion of the plunger rod can be relatively small, such as comprising structure inside the stopper.

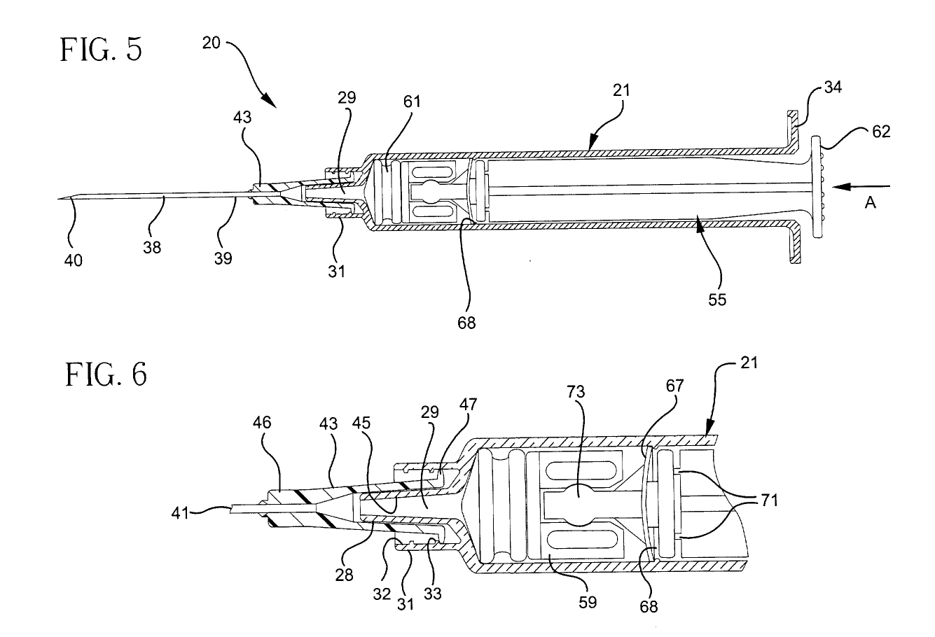

The present embodiment also includes a catch on proximal portion 58 of the plunger rod and a discontinuity on the inside surface 23 of the fluid chamber. In this embodiment, the discontinuity is an annular inwardly directed rib 68. The catch and the discontinuity are positioned and configured so that upon further distal movement of the proximal portion of the plunger rod after disengagement of the disengageable connection, catch 67 will engage discontinuity 68 to lock proximal portion 58 of the plunger rod in the barrel as best illustrated in FIGS. 5 and 6. The discontinuity can project from the inside surface into the chamber such as annular rib 68. The projection does not have to be continuous but may be one or more discreet projections from the inside surface. The discontinuity may also be a recess or indentation which is annular or on selected portions of the inside surface or any step in the inside of the barrel, such as a change in the inside diameter of the barrel. The catch accordingly will be configured to react appropriately with the discontinuity. If this discontinuity is a recess the catch must be larger than the inside diameter of the barrel and it will expand into the recess when the proximal and distal portions disconnect and the proximal portion is appropriately positioned to align the catch and the discontinuity.

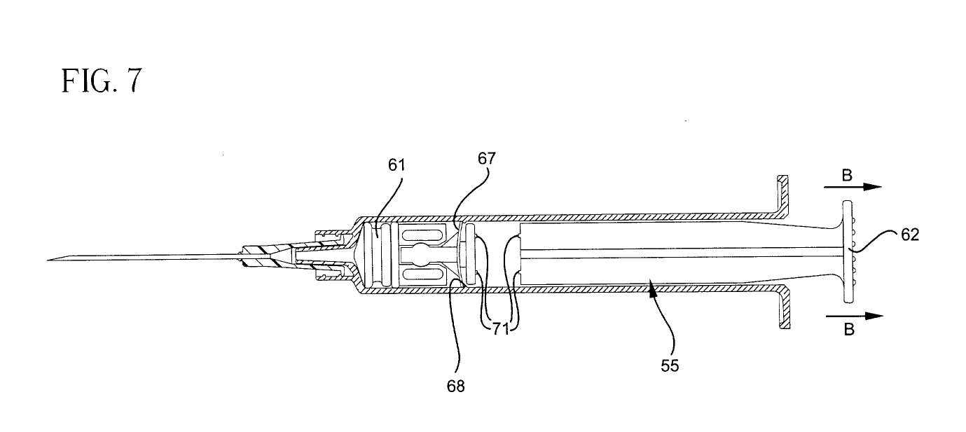

In use, the syringe of the present invention can be filled from a vial, ampoule or other suitable container using known safe procedures. An important advantage of the present invention is that the plunger rod can be moved back and forth along the barrel as many times as necessary to properly fill the chamber. For example, the syringe barrel may be filled with sterile water and then the sterile water can be injected into a vial containing a lyophilized medication which is then drawn back into the syringe barrel. Many single-use syringes in the prior art only allow one or two proximal motions of the plunger rod with respect to the barrel. With these single-use syringes, once the plunger is moved in a distal direction with respect to the barrel it can no longer be withdrawn. Therefore, mixing sterile water and a lyophilized medication as described above is not possible. Another advantage of the present invention is that the plunger can be moved to its distal-most position with respect to the barrel and then moved proximally. Some prior art syringes automatically block the plunger to the barrel when the plunger is moved to its distal-most position. These prior art designs can lead to unintentional locking of the plunger before use and can compromise filling and mixing procedures. The liquid in the barrel can now be injected into a patient or delivered in another suitable manner such as through the pierceable septum of a catheter connector. Upon completion of the injection, the user can apply an additional force, indicated as A in FIG. 5, to the proximal portion. In this embodiment, the breakable connections are broken by the application of force A applied to the proximal portion. Force A is sufficient to disengage the disengageable connection which causes the plunger rod to separate into two unusable pieces. After disengaging the disengageable connection, the proximal portion of the plunger rod moves distally so that catch 67 may now engage rib 68. To further prevent any attempt to disassemble the syringe for the improper purpose of altering it to be reused again. Without the catch and the rib the proximal portion of the plunger rod would be easily removable and a tool may be used to draw the distal portion out of the barrel so that the barrel can be used with another plunger rod or the portions can be reassembled for additional use. With the present invention all of the components are locked in the barrel discouraging attempts to reuse the syringe and minimizing contamination, and also making waste disposal easier by keeping the syringe from coming apart. This is an important advantage of the present invention over the prior art. It not only provides a breakable connection to separate the distal and proximal portions of the plunger rod but it also includes means for retaining the proximal end of the plunger rod in the barrel after separation of the proximal and distal ends of the plunger rod. This embodiment also includes a second disengageable connection on proximal portion 58 of the plunger rod. This disengageable connection is positioned proximal to catch 67 and is strong enough to hold the proximal portion together during normal use of the syringe and disengageable upon application of an additional force along the longitudinal axis to dislodge the plunger from the barrel. In this embodiment the second disengageable connection comprises one or more frangible links 71 which will break upon application of an excessive force, such as force B in FIG. 7, to improperly disassemble the syringe for the purpose of reuse. Upon breaking of the frangible links the distal portion of the plunger rod is still locked in the distal end of the barrel by virtue of the action of the catch and the rib. As with the disengageable connection which holds the proximal and distal portions of the plunger, the second disengageable connection may be breakable or disengageable as through a snap-fit arrangement. The second disengageable connection may be formed by integrally molding all elements of the proximal portion of the plunger rod or by using separate elements with adhesives, frangible materials and the like to achieve the desired result.

Accordingly, the present embodiment contains three anti-mis-use features which are: means for separating the proximal and distal ends of the plunger upon completion of the injection; means for locking the proximal portion of the plunger in the barrel after completion of the injection; and means for allowing the proximal portion of the plunger rod to come apart before the engagement of the catch in the rib is overcome, thus keeping the distal portion of the plunger rod locked in the barrel after the injection process is complete.

The present embodiment also includes means for controlling the distal motion of the proximal portion of the plunger rod after disengagement of the disengageable connection. In this embodiment the means for controlling motion comprises an enlarged section 73 on distal projection 64, and a flexible restriction 74 on distal portion 59. Upon the application of force A to disengage the disengageable connection, the proximal portion of the plunger rod moves distally. This distal motion can be unrestricted and immediate or it can be restrained to provide a smoother less abrupt transition after the disengageable connection is disengaged. This restraint is provided by enlarged section 73 acting with flexible restriction 74. As the proximal portion moves distally the enlarged section flexes the restriction using energy and slowing the distal motion of the plunger rod. Any structure that will absorb energy and decelerate the distal motion of the proximal portion may be used to achieve this purpose.

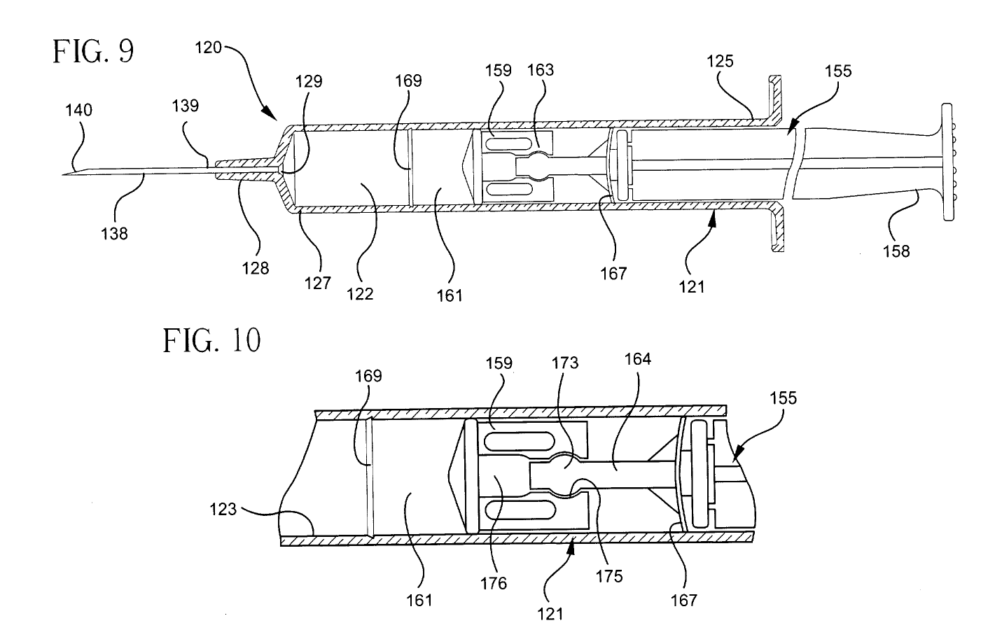

FIGS. 9-10 illustrate an alternative embodiment of the present invention. In this embodiment, a syringe 120 comprises a barrel 121 including a fluid chamber 122 having an inside surface 123, an open proximal end 125 and a distal end 127 having a passageway 129 therethrough in fluid communication with the chamber. The barrel further includes an elongate distal tip 128 extending from the distal end wherein passageway 129 extends through the tip. A cannula 138 having a proximal end 139, a distal end 40 and a lumen (not shown) therethrough. The proximal end of the cannula is attached to distal tip 126 so that the lumen of the cannula is in fluid communication with the passageway of the barrel.

A plunger rod 155 having a proximal portion 158 and a distal portion 159 connected by a disengageable connection 163. Distal portion 159 includes a rigid stopper 161 positioned in fluid-tight engagement with the inside surface of the chamber. The stopper is preferably integrally molded with the distal portion of the plunger rod. This embodiment functions in a substantially similar manner to the embodiment of FIGS. 1-8. As with the embodiment of FIGS. 1-8, the disengageable connection is strong enough to hold the proximal portion and the distal portion together during normal use of the syringe and disengageable upon application of an additional force applied to the proximal portion. In this embodiment the disengageable connection comprises an enlarged section 173 on distal projection 164 and a deflectable engaging recess 175 in distal portion 159. Upon completion of the injection process and application of an additional distally directed force to the plunger rod, enlarged section 173 deflects recess 175 and moves into open clearance cavity 176 in the distal portion. A catch 167 on the proximal portion 158, and a discontinuity in the form of an annular groove 169 in the chamber are sized and positioned so that upon further distal movement of the proximal portion, after disengagement of the disengageable connection, the catch engages the annular groove to lock the proximal portion of the plunger rod in the barrel. In this embodiment, the catch may include any projection which extends radially to a distance far enough to engage the annular groove and is flexible enough to allow the plunger rod to be cycled within the barrel without locking the plunger rod to the barrel before engaging the annular groove. Likewise, the annular groove merely represents numerous forms of recesses continuous and discontinuous which can be placed in the barrel for engaging the catch.

The present invention is a significant advance over single-use syringes of the prior art. In particular, it allows multiple strokes of the plunger with respect to the barrel without automatically locking or rendering the syringe unusable. It also allows the plunger rod to move to its distal-most position inside the barrel without automatically locking the plunger rod to the barrel. It also provides a mechanism for prevent or discourage re-use. The plunger rod is breakable so that its proximal and distal portions are separated and the syringe can no longer be used to inject medication. Structure is also provided to prevent removal of the proximal portion of the plunger from the barrel after disengagement of the disengageable connection between the proximal portion and the distal portion of the plunger rod. Structure is also provided to allow the proximal portion of the plunger rod to break in response to an attempt to remove the plunger rod from the barrel after the injection process is complete. Further, structure is provided for controlling the distal motion of the proximal portion of the plunger rod after disengagement of the disengageable connection.

What is claimed is:

1. A syringe comprising:
a barrel including a fluid chamber having an inside surface, an open proximal end, and a distal end having a passageway therethrough in fluid communication with said chamber;
a plunger rod having a longitudinal axis, a proximal portion and a distal portion connected by a disengageable connection, said distal portion including a stopper slidably positioned in fluid-tight engagement with said inside surface of said chamber for drawing fluid into and out of said chamber by movement of said plunger rod relative to said barrel;
said disengageable connection being strong enough to hold said proximal portion and said distal portion together during normal use of said syringe and disengageable upon application of an additional force applied to said proximal portion along said longitudinal axis; and
a catch on said proximal portion of said plunger rod and a discontinuity in said chamber positioned so that upon further distal movement of said proximal portion, after disengagement of said disengageable connection, said catch engages said discontinuity to lock said proximal portion of said plunger rod in said barrel.

2. The syringe of claim 1 further including a second disengageable connection on said proximal portion of said plunger rod proximal to said catch, said second disengageable connection being strong enough to hold said proximal portion together during normal use of said syringe and disengageable upon application of an additional force along said longitudinal axis to dislodge said plunger rod from said barrel.

3. The syringe of claim 1 wherein one of said proximal portion and said distal portion includes an axial projection having at least one transverse protuberance projecting therefrom, said protuberance being connected to the other of said proximal portion and said distal portion, said disengageable connection being a breakable connection on said protuberance.

4. The syringe of claim 3 wherein said axial projection includes a plurality of transverse protuberances.

5. The syringe of claim 4 wherein said transverse protuberances project from opposite sides of said axial projection.

6. The syringe of claim 3 wherein said proximal portion, said distal portion and said breakable connection are integrally molded of plastic material.

7. The syringe of claim 3 wherein said breakable connection is made of material selected form the group of polyethylene, polystyrene, polypropylene and adhesives.

8. The syringe of claim 1 wherein said disengageable connection comprises:
one of said proximal portion and said distal portion including a recess and the other of said proximal portion and said distal portion including a projection releasably engaging said recess in a snap-fit arrangement.

9. The syringe of claim 1 wherein said catch is a radial projection on said proximal portion.

10. The syringe of claim 9 wherein said catch is a flange on said proximal portion.

11. The syringe of claim 1 wherein said catch is a recess in said proximal portion.

12. The syringe of claim 1 wherein said discontinuity is a recess in said inside surface of said chamber.

13. The syringe of claim 1 wherein said discontinuity is a projection on said inside surface of said chamber.

14. The syringe of claim 2 wherein said second disengageable connection comprises a frangible link formed by an area of reduced cross-sectional area along said longitudinal axis of said proximal portion.

15. The syringe of claim 1 further including means for controlling the distal motion of said proximal portion after disengagement of said disengageable connection.

16. The syringe of claim 15 wherein said means for controlling comprises a flexible restriction in said distal portion which is deflected by said proximal portion when said disengageable connection is disengaged.

17. The syringe of claim 1 further including an elongate distal tip extending from said distal end of said barrel wherein said passageway extends through said tip.

18. The syringe of claim 17 further including a needle assembly including a cannula having a proximal end, a distal end and a lumen therethrough, a hub having an open proximal end with a cavity therein, and a distal end joined to said proximal end of said cannula so that said lumen is in fluid communication with said cavity, said needle assembly being connected to said barrel so that said elongate tip of said barrel is in said cavity of said hub.

19. The syringe of claim 1 further including a cannula having a proximal end a distal end and a lumen therethrough, said proximal end of said cannula being joined to said distal end of said barrel so that said lumen is in fluid communication with said passageway.

20. The syringe of claim 1 wherein said stopper and said distal portion are integrally molded of plastic material.

21. A syringe comprising:
a barrel having a fluid chamber, an open proximal end, a distal end and an elongate tip extending from said distal end having a passageway therethrough in fluid communication with said chamber;
a plunger rod having a longitudinal axis, a proximal portion and a distal portion connected by a disengageable connection, said disengageable connection being a breakable connection, said distal portion including a stopper slidably positioned in fluid-tight engagement with said inside surface of said chamber for drawing fluid into and out of said chamber by movement of said plunger rod relative to said barrel; said disengageable connection being strong enough to hold said proximal portion and said distal portion together during normal use of said syringe and disengageable upon application of an additional force applied to said proximal portion along said longitudinal axis; and
an annular flange on said proximal portion of said plunger rod and a discontinuity in said chamber positioned so that upon further additional movement of the proximal portion, after disengagement of said disengageable connection, said flange engages said discontinuity to lock said proximal portion of said plunger rod in said barrel.

22. The syringe of claim 21 further including a second disengageable connection on said proximal portion of said plunger rod proximal to said catch, said second disengageable connection being strong enough to hold said proximal portion together during normal use of said syringe and disengageable upon application of an additional force along said longitudinal axis to dislodge said plunger from said barrel.
